(19) United States
(12) Patent Application Publication
(10) Pub. No.: US 2025/0206756 A1
(43) Pub. Date: Jun. 26, 2025

(54) INHIBITORS

(71) Applicant: Pfizer Inc., New York, NY (US)

(72) Inventors: Brian Stephen Gerstenberger, Cambridge, MA (US); Carley Jeanne Sue Heck, New London, CT (US); Manjinder Singh Lall, East Lyme, CT (US); Mihir Dineshkumar Parikh, Greenwich, RI (US); Usa Reilly, West Haven, CT (US); Gwenaella Christine Rescourio, San Diego, CA (US)

(73) Assignee: Pfizer Inc., New York, NY (US)

(21) Appl. No.: 18/987,343

(22) Filed: Dec. 19, 2024

Related U.S. Application Data
(60) Provisional application No. 63/613,457, filed on Dec. 21, 2023.

(51) Int. Cl. C07D 513/10; A61K 31/438

(52) U.S. Cl. CPC C07D 513/10; A61K 31/438

(57) ABSTRACT

Described herein are novel compounds in an isolated or crystalline form, pharmaceutical compositions and methods for the treatment of and prevention of various disorders, in particular, skin disorders like acne.

### FIELD OF THE INVENTION

### Field of the Invention

The present invention relates to novel Acetyl CoA-Carboxylase (ACC) inhibitors, pharmaceutical compositions comprising such compounds and their use as medicaments. More particularly, the present invention provides novel ACC inhibitors which are useful for the treatment of and prevention of acne.

### Background

Current standard of care for acne includes topical therapies for mild to moderate disease, and systemic therapy for moderate to severe disease. These current therapies are either marginally effective or lack suitable safety profiles for widespread use. Topical acne treatments include retinoids, topical antibiotics, benzoyl peroxides and combinations thereof. Systemic treatments include hormonal therapies, oral antibiotics and isotretinoin (Dawson et al., BMJ 2013; 346:12634). Hormonal therapies, including oral contraceptives and androgen receptor blockers, are used in female patients for the treatment of moderate to severe acne with modest efficacy. Oral antibiotics including doxycycline, minocycline, tetracycline and erythromycin are also modestly effective in treating acne, particularly when matched against patterns of C. acnes resistance; although, photosensitivity and gastrointestinal disturbance limit their use (Gannon et al., Family Pract. 2011; 60:290-92). Isotretinoin, although highly efficacious, presents a number of serious adverse effects. The agent is highly teratogenic and requires special prescribing precautions and routine pregnancy testing. Additionally, isotretinoin causes severe mucocutaneous toleration issues (dry skin, eyes, nasal passages, lips, etc.) which can be dose limiting if not adequately managed with palliative care. Isotretinoin treatment is associated with adverse plasma lipid changes (increased TG, LDL) and hepatic toxicity (ALT/AST elevation requiring liver function testing prior to treatment. Additionally, isotretinoin therapy has also been associated with myalgia (50% of patients have elevated CK levels), calcification of ligaments and detrimental ocular effects (loss of night vision, loss of color vision and eye dryness). In isolated cases, isotretinoin has been associated with neurological/psychological adverse effects including depression, psychosis and potentially suicide.

Therefore, a need exists for a novel approach to treating acne with a favorable efficacy/safety profile. The present invention provides a new therapeutic approach for treating acne comprising the use of ACC inhibitors. There is thus a need to provide new compounds that are potent, selective inhibitors of sebum secretion with suitable pharmacokinetic properties, particularly compounds which can be administered by topical administration and are efficacious in the treatment of acne.

New compounds which are ACC inhibitors with selective efficacy are needed which provide an alternative to current usage.

Recently identified spiro(benzo[d]thiazole) derived compounds have been described in commonly assigned U.S. Ser. No. 63/393,712, the contents of which are incorporated herein by reference in their entirety, having the following generic structural formula:

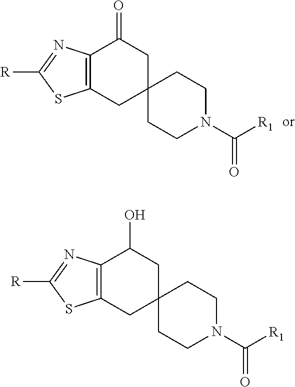

Such compounds have significant ACC activity, and may be useful therapeutically for the treatment of a variety of disorders including acne. Novel metabolites of such compounds have now been discovered by the present inventors that are highly active as inhibitors of ACC, and surprisingly are capable of greater therapeutic index and accordingly, have the potential to be superior pharmaceutical agents for the treatment of ACC-mediated disorders.

### SUMMARY OF THE INVENTION

The present invention relates to spiro(benzo[d]thiazole) compounds and isolated forms of such compounds. The isolated compounds of the invention have been found to possess unexpectedly advantageous activity including high efficacy and likely reduced incidence of adverse effects. The isolated compounds may be employed in pharmaceutical compositions in combination with pharmaceutically acceptable excipients and in methods of treating conditions mediated by ACC.

The present invention provides a compound of formula I having the structure:

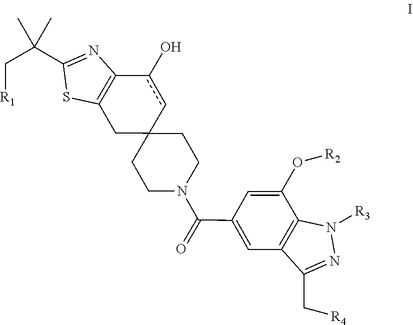

or a pharmaceutically acceptable salt, wherein R₁ is H or OH, R₂ is OH or ethyl, R₃ is H or methyl, and R₄ is H or OH; and, wherein the dashed line may be present or absent, and if present, indicates a double bond.

In other aspects, the present invention also provides a compound of formula I or a pharmaceutically acceptable salt in an isolated form.

In other aspects, the present invention also provides pharmaceutical compositions which comprise a pharmaceutically acceptable excipient and a compound of formula I or a pharmaceutically acceptable salt thereof.

Arthritis, including rheumatoid arthritis, juvenile arthritis, and psoriatic arthritis;

Autoimmune or inflammatory diseases or disorders, including Hashimoto's thyroiditis, autoimmune hemolytic anemia, autoimmune atrophic gastritis of pernicious anemia, autoimmune encephalomyelitis, autoimmune orchitis, Goodpasture's disease, autoimmune thrombocytopenia, sympathetic ophthalmia, myasthenia gravis, Graves' disease, primary biliary cirrhosis, autoimmune hepatitis, primary sclerosing cholangitis, chronic aggressive hepatitis, non-alcoholic fatty liver disease, non-alcoholic steatohepatitis ulcerative colitis and membranous glomerulopathy, systemic lupus erythematosus, rheumatoid arthritis, psoriatic arthritis, Sjogren's syndrome, Reiter's syndrome, polymyositis, dermatomyositis, type I interferonopathies including Aicardi-Goutibres syndrome and other mendelian diseases of overexpression of type I interferon systemic sclerosis, polyarteritis nodosa, multiple sclerosis, relapsing remitting multiple sclerosis, primary progressive multiple sclerosis, secondary progressive multiple sclerosis, and bullous pemphigoid, and additional autoimmune diseases, which can be O-cell (humoral) based or T-cell based, including Cogan's syndrome, ankylosing spondylitis, Wegener's granulomatosis, autoimmune alopecia, Type I or juvenile onset diabetes, or thyroiditis;

Cancers or tumors, including alimentary/gastrointestinal tract cancer, colon cancer, liver cancer, skin cancer including mast cell tumor and squamous cell carcinoma, breast and mammary cancer, ovarian cancer, prostate cancer, lymphoma, leukemia, including acute myelogenous leukemia and chronic myelogenous leukemia, kidney cancer, lung cancer, muscle cancer, bone cancer, bladder cancer, brain cancer, melanoma including oral and metastatic melanoma, Kaposi's sarcoma, myelomas including multiple myeloma, myeloproliferative disorders, proliferative diabetic retinopathy, or angiogenic-associated disorders including solid tumors;

Diabetes, including Type I diabetes or complications from diabetes;

Eye diseases, disorders or conditions including autoimmune diseases of the eye, keratoconjunctivitis, vernal conjunctivitis, uveitis including uveitis associated with Behcet's disease and lens-induced uveitis, keratitis, herpetic keratitis, conical keratitis, corneal epithelial dystrophy, keratoleukoma, ocular premphigus, Mooren's ulcer, scleritis, Grave's ophthalmopathy, Vogt-Koyanagi-Harada syndrome, keratoconjunctivitis sicca (dry eye), phlyctenule, iridocyclitis, sarcoidosis, endocrine ophthalmopathy, sympathetic ophthalmitis, allergic conjunctivitis, or ocular neovascularization;

Intestinal inflammations, including Crohn's disease, ulcerative colitis, inflammatory bowel disease, celiac diseases, proctitis, eosinophilic gastroenteritis, or mastocytosis;

Neurodegenerative diseases including motor neuron disease, Alzheimer's disease, Parkinson's disease, amyotrophic lateral sclerosis, Huntington's disease, cerebral ischemia, or neurodegenerative disease caused by traumatic injury, strike, glutamate neurotoxicity or hypoxia; ischemic/reperfusion injury in stroke, myocardial ischemia, renal ischemia, heart attacks, cardiac hypertrophy, atherosclerosis and arteriosclerosis, organ hypoxia, or platelet aggregation;

Skin diseases, conditions or disorders including atopic dermatitis, hand dermatitis, contact dermatitis, allergic contact dermatitis, irritant contact dermatitis, neurodermatitis, perioral dermatitis, stasis dermatitis, dyshidrotic eczema, xerotic dermatitis, nummular dermatitis, seborrheic dermatitis, eyelid dermatitis, diaper dermatitis, dermatomyositis, lichen planus, lichen sclerosis, alopecia areata, vitiligo, rosacea, epidermolysis bullosa, keratosis pilaris, pityriasis alba, pemphigus, vulvovaginitis, acne, chronic spontaneous urticaria, chronic idiopathic urticaria, chronic physical urticaria, vogt-koyanagi-harada disease, sutton nevus/nevi, post inflammatory hypopigmentation, senile leukoderma, chemical/drug-induced leukoderma, cutaneous lupus erythematosus, discoid lupus, palmoplantar pustulosis, pemphigoid, sweet's syndrome, hidradenitis suppurativa, psoriasis, plaque psoriasis, pustular psoriasis, nail psoriasis, flexural psoriasis, guttate psoriasis, psoriatic arthritis, erythrodermic psoriasis, or inverse psoriasis;

Allergic reactions including allergic dermatitis in mammal (including horse allergic diseases such as bite hypersensitivity), summer eczema, sweet itch in horses, heaves, inflammatory airway disease, recurrent airway obstruction, airway hyper-responsiveness, or chronic obstruction pulmonary disease;

Asthma and other obstructive airways diseases, including chronic or inveterate asthma, late asthma, bronchitis, bronchial asthma, allergic asthma, intrinsic asthma, extrinsic asthma, or dust asthma; and,

Transplant rejection, including pancreas islet transplant rejection, bone marrow transplant rejection, graft-versus-host disease, organ and cell transplant rejection such as bone marrow, cartilage, cornea, heart, intervertebral disc, islet, kidney, limb, liver, lung, muscle, myoblast, nerve, pancreas, skin, small intestine, or trachea, or xeno transplantation.

In another aspect the present invention also provides methods for the preparation of compounds of the present invention.

The present invention will be further understood from the following description given by way of example only. While the present invention is not so limited, an appreciation of various aspects of the invention will be gained through the following discussion and the examples.

The term “subject” refers to human, livestock or companion animals.

The term “treating” or “treatment” means an alleviation of symptoms associated with a disease, disorder or condition, or halt of further progression or worsening of those symptoms.

Depending on the disease and condition of the subject, the term “treatment” as used herein may include one or more of curative, palliative and prophylactic treatment. Treatment can also include administering a pharmaceutical formulation of the present invention in combination with other therapies.

The term “therapeutically effective” indicates the capability of an agent to prevent, or improve the severity of, the disorder, while avoiding adverse side effects typically associated with alternative therapies. The phrase “therapeutically effective” is to be understood to be equivalent to the phrase “effective for the treatment, prevention, or amelioration”, and both are intended to qualify the amount of each agent for use in the combination therapy which will achieve the goal of improvement in the severity of cancer, cardiovascular disease, or pain and inflammation and the frequency of incidence over treatment of each agent by itself, while avoiding adverse side effects typically associated with alternative therapies.

“Pharmaceutically acceptable” means suitable for use in a subject.

If substituents are described as being “independently selected” from a group, each substituent is selected independent of the other. Each substituent therefore may be identical to or different from the other substituent(s). In some embodiments, the metabolite compounds, or salts thereof, are substantially isolated.

The terms “isolated”, “purified”, “in purified form”, “in isolated form” or “purified substance” means that a compound, or salt thereof, for a compound refers to the physical state of the compound after being isolated from a synthetic process, e.g., from a reaction mixture. Thus the terms “isolated”, “purified”, “in purified form”, “in isolated form” or “purified substance” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan. As examples, the purification techniques disclosed herein (e.g., LC-MS and LC-MS/MS techniques) result in isolated forms of the subject compounds. Such isolation and purification techniques would be expected to result in product purities containing at least about 70%, at least about 80%, at least about 90%, at least about 95%, at least about 97%, or at least about 99% by weight of the compound, or salt thereof.

### DETAILED DESCRIPTION OF THE INVENTION

The present invention is related to compounds which are selective ACC modulators useful for the treatment of diseases and conditions associated with dysregulation of the ACC. The present invention further provides isolated compounds which are selective ACC modulators useful for the treatment of diseases and conditions associated with dysregulation of the ACC. The present invention also provides prodrugs and pharmaceutical compositions comprising such ACC modulators as well as methods of treating and/or preventing such diseases and conditions.

In a first aspect the invention provides a compound of formula I having the structure:

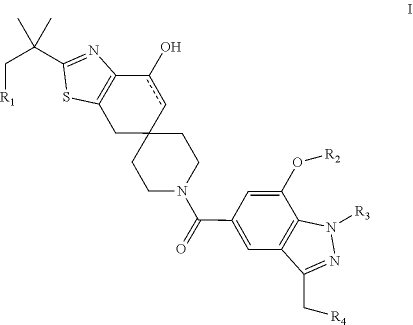

or a pharmaceutically acceptable salt, wherein R₁ is H or OH, R₂ is OH or ethyl, R₃ is H or methyl, and R₄ is H or OH; and, wherein the dashed line may be present or absent, and if present, indicates a double bond.

Described below are embodiments (E) of this first aspect of the invention where, for convenience, E1 is identical thereto.
E1. A compound of formula I having the structure:

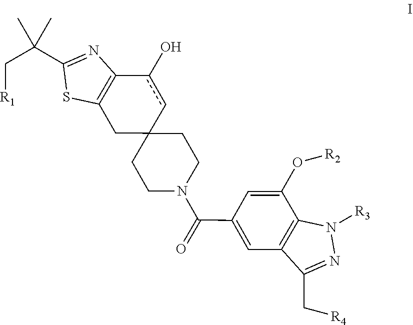

or a pharmaceutically acceptable salt, wherein R₁ is H or OH, R₂ is OH or ethyl, R₃ is H or methyl, and R₄ is H or OH; and, wherein the dashed line may be present or absent, and if present, indicates a double bond.
E2. The compound according to E1 or a pharmaceutically acceptable salt thereof, wherein R₁ is H, R₂ is ethyl; R₃ is H, and R₄ is H.
E3. The compound according to E1 or a pharmaceutically acceptable salt thereof, wherein R₁ is OH, R₂ is ethyl; R₃ is methyl, and R₄ is H.
E4. A compound of formula I having the structure:

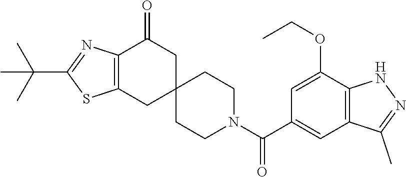 [SMILES: CCOc1cc(C(=O)N2CCC3(CC2)CC(=O)c2nc(C(C)(C)C)sc2C3)cc2c(C)n[nH]c12]

or a pharmaceutically acceptable salt thereof.
E5. A compound of formula I having the structure:
or a pharmaceutically acceptable salt thereof.

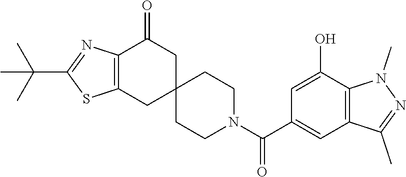 [SMILES: Cc1nn(C)c2c(O)cc(C(=O)N3CCC4(CC3)CC(=O)c3nc(C(C)(C)C)sc3C4)cc12]

E6. A compound of formula I having the structure:

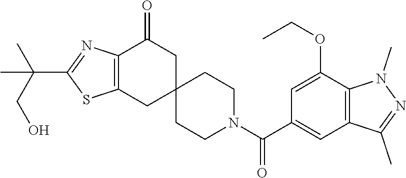 [SMILES: CCOc1cc(C(=O)N2CCC3(CC2)CC(=O)c2nc(C(C)(C)CO)sc2C3)cc2c(C)nn(C)c12]

or a pharmaceutically acceptable salt thereof.
E7. A compound of formula I having the structure:

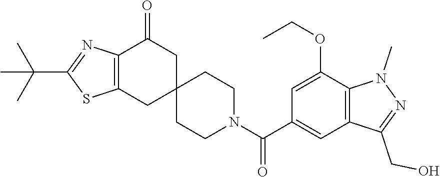 [SMILES: CCOc1cc(C(=O)N2CCC3(CC2)CC(=O)c2nc(C(C)(C)C)sc2C3)cc2c(CO)nn(C)c12]

or a pharmaceutically acceptable salt thereof.
E8. The compound according to E1 selected from the group consisting of:
2-(tert-butyl)-1′-(7-ethoxy-3-methyl-1H-indazole-5-carbonyl)-5H-spiro[benzo[d]thiazole-6,4′-piperidin]-4(7H)-one;
2-(tert-butyl)-1′-(7-hydroxy-1,3-dimethyl-1H-indazole-5-carbonyl)-5H-spiro[benzo[d]thiazole-6,4′-piperidin]-4(7H)-one;
1′-(7-ethoxy-1,3-dimethyl-1H-indazole-5-carbonyl)-2-(1-hydroxy-2-methylpropan-2-yl)-5H-spiro[benzo[d]thiazole-6,4′-piperdin]-4(7H)-one;
rac-(R)-(2-(tert-buty)-4-hydroxy-4,7-dihydro-5H-spiro[benzo[d]thiazole-6,4′-piperdin]-1′-yl)(7-ethoxy-1,3-dimethyl-1H-indazol-5-yl)methanone;
2-(tert-butyl)-1′-(7-ethoxy-3-(hydroxymethyl)-1-methyl-1H-indazole-5-carbonyl)-5H-spiro[benzo[d]thiazole-6,4′-piperdin]-4(7H)-one;
rac-(2-(tert-butyl)-4-hydroxy-4,7-dihydro-5H-spiro[benzo[d]thiazole-6,4′-piperidin]-1′-yl)(7-ethoxy-3-methyl-1H-indazol-5-yl)methanone;
1′-(7-ethoxy-3-methyl-1H-indazole-5-carbonyl)-2-(1-hydroxy-2-methylpropan-2-yl)-5H-spiro[benzo[d]thiazole-6,4′-piperdin]-4(7H)-one;
1′-(7-hydroxy-3-methyl-1H-indazole-5-carbonyl)-2-(1-hydroxy-2-methylpropan-2-yl)-5H-spiro[benzo[d]thiazole-6,4′-piperdin]-4(7H)-one; or,
   a pharmaceutically acceptable salt thereof.
   E9. The compound according to E1 wherein the compound is 2-(tert-butyl)-1′-(7-ethoxy-3-methyl-1H-indazole-5-carbonyl)-5H-spiro[benzo[d]thiazole-6,4′-piperidin]-4(7H)-one; or, a pharmaceutically acceptable salt thereof.
   E10. The compound according to E1 wherein the compound is 2-(tert-butyl)-1′-(7-hydroxy-1,3-dimethyl-1H-indazole-5-carbonyl)-5H-spiro[benzo[d]thiazole-6,4′-piperdin]-4(7H)-one; or, a pharmaceutically acceptable salt.
   E11. The compound according to E1 wherein the compound is 1′-(7-ethoxy-1,3-dimethyl-1H-indazole-5-carbonyl)-2-(1-hydroxy-2-methylpropan-2-yl)-5H-spiro[benzo[d]thiazole-6,4′-piperidin]-4(7H)-one; or, a pharmaceutically acceptable salt.
   E12. The compound according to E1 wherein the compound is rac-(R)-(2-(tert-butyl-4-hydroxy-4,7-dihydro-5H-spiro[benzo[d]thiazole-6,4′-piperdin]-1′-yl)(7-ethoxy-1,3-dimethyl-1H-indazol-5-yl)methanone; or, a pharmaceutically acceptable salt.
   E13. The compound according to E1 wherein the compound is 2-(tert-butyl)-1′-(7-ethoxy-3-(hydroxymethyl)-1-methyl-1H-indazole-5-carbonyl)-5H-spiro[benzo[d]thiazole-6,4′-piperidin]-4(7H)-one; or, a pharmaceutically acceptable salt.
   E14. The compound according to E1 wherein the compound is rac-(2-(tert-butyl)-4-hydroxy-4,7-dihydro-5H-spiro[benzo[d]thiazole-6,4′-piperidin]-1′-yl)(7-ethoxy-3-methyl-1H-indazol-5-yl)methanone; or, a pharmaceutically acceptable salt.
   E15. The compound according to E1 wherein the compound is 1′-(7-ethoxy-3-methyl-1H-indazole-5-carbonyl)-2-(1-hydroxy-2-methylpropan-2-yl)-5H-spiro[benzo[d]thiazole-6,4′-piperidin]-4(7H)-one; or, a pharmaceutically acceptable salt.
   E16. The compound according to E1 wherein the compound is 1′-(7-hydrooxy-1,3-dimethyl-1H-indazole-5-carbonyl)-2-(1-hydroxy-2-methylpropan-2-yl)-5H-spiro[benzo[d]thiazole-6,4′-piperidin]-4(7H)-one; or, a pharmaceutically acceptable salt.
   E17. The compound or a pharmaceutically acceptable salt thereof according to any one of E1 to E16 in an isolated form.
   E18. The compound or a pharmaceutically acceptable salt thereof according to any one of E1 to E16, in crystalline form.
   E19. A pharmaceutical composition comprising a compound according to any one of E1 to E16, or a pharmaceutically acceptable salt thereof, and a pharmaceutically acceptable excipient.
   E20. A method of treating or preventing a disease or condition for which a ACC inhibitor is indicated, in a subject in need of such treatment, comprising administering to the subject a therapeutically effective amount of a compound according to any one of E1 to E16, or a pharmaceutically acceptable salt thereof.
   E21. A method of treating or preventing an inflammatory or autoimmune condition comprising administering to a subject in need thereof a therapeutically effective amount of a compound according to any one of E1 to E16, or a pharmaceutically acceptable salt thereof.
   E22. A method of treating or preventing a disease or condition selected from inflammation, autoimmune disease, neuroinflammation, arthritis, rheumatoid arthritis, spondyloarthropathies, systemic lupus erythematous, lupus nephritis, osteoarthritis, gouty arthritis, pain, fever, pulmonary sarcoidosis, silicosis, cardiovascular disease, atherosclerosis, myocardial infarction, thrombosis, congestive heart failure and cardiac reperfusion injury, cardiomyopathy, stroke, ischemia, reperfusion injury, brain edema, brain trauma, neurodegeneration, liver disease, inflammatory bowel disease, Crohn's disease, ulcerative colitis, nephritis, retinitis, retinopathy, macular degeneration, glaucoma, diabetes (type 1 and type 2), diabetic neuropathy, viral and bacterial infection, myalgia, endotoxic shock, toxic shock syndrome, osteoporosis, multiple sclerosis, endometriosis, menstrual cramps, vaginitis, candidiasis, cancer, fibrosis, obesity, muscular dystrophy, polymyositis, dermatomyositis, autoimmune hepatitis, primary biliary cirrhosis, primary sclerosing cholangitis, vitiligo, Alzheimer's disease, skin flushing, eczema, psoriasis, atopic dermatitis, sunburn, keloid, hypertrophic scar, rheumatic diseases, urticaria, discoid lupus, cutaneous lupus, central nervous system lupus, psoriatic arthritis, asthma, allergic asthma, type I interferonopathies including Aicardi-Goutibres syndrome and other mendelian diseases of overexpression of type I interferon, primary progressive multiple sclerosis, relapsing remitting multiple sclerosis, non-alcoholic fatty liver disease, non-alcoholic steatohepatitis, scleroderma, alopecia areata, scarring alopecia, prurigo, prurigo nodularis, CPUO, lichen diseases, lichen planus, Steven's Johnson's syndrome, spondylopathy, myositis, vasculitis, pemphigus, lupus, major depression disorder, allergy, dry eye syndrome, transplant rejection, cancer, septic shock, cardiopulmonary dysfunction, acute respiratory disease, ankylosing spondylitis, cachexia, chronic graft-versus-host disease, acute graft-versus-host disease, Celiac Sprue, idiopathic thrombocytopenic thrombotic purpura, thrombotic thrombocytopenic purpura, myasthenia gravis, Sjogren's syndrome, epidermal hyperplasia, cartilage inflammation, bone degradation, juvenile arthritis, juvenile rheumatoid arthritis, pauciarticular juvenile rheumatoid arthritis, polyarticular juvenile rheumatoid arthritis, systemic onset juvenile rheumatoid arthritis, juvenile ankylosing spondylitis, juvenile enteropathic arthritis, juvenile Reter's Syndrome, SEA Syndrome, juvenile dermatomyositis, juvenile psoriatic arthritis, juvenile scleroderma, juvenile systemic lupus erythematosus, juvenile vasculitis, pauciarticular rheumatoid arthritis, polyarticular rheumatoid arthritis, systemic onset rheumatoid arthritis, enteropathic arthritis, reactive arthritis, Reter's Syndrome, myolitis, polymyolitis, dermatomyolitis, polyarteritis nodosa, Wegener's granulomatosis, arteritis, polymyalgia rheumatica, sarcoidosis, sclerosis, primary biliary sclerosis, sclerosing cholangitis, dermatitis, Still's disease, chronic obstructive pulmonary disease, Guillain-Barre disease, Graves' disease, Addison's disease, Raynaud's phenomenon, psoriatic epidermal hyperplasia, plaque psoriasis, guttate psoriasis, inverse psoriasis, pustular psoriasis, erythrodermic psoriasis, an immune disorder associated with or arising from activity of pathogenic lymphocytes, noninfectious uveitis, Behcet's disease and Vogt-Koyanagi-Harada syndrome, comprising administering to a subject in need thereof a therapeutically effective amount of a compound according to any one of E1 to E16, or a pharmaceutically acceptable salt thereof.
   E23. The method according to E22 wherein the compound is selected from the group consisting of:
2-(tert-butyl)-1′-(7-ethoxy-3-methyl-1H-indazole-5-carbonyl)-5H-spiro[benzo[d]thiazole-6,4′-piperidin]-4(7H)-one;
2-(tert-butyl)-1′-(7-hydroxy-1,3-dimethyl-1H-indazole-5-carbonyl)-5H-spiro[benzo[d]thiazole-6,4′-piperidin]-4(7H)-one;
1′-(7-ethoxy-1,3-dimethyl-1H-indazole-5-carbonyl)-2-(1-hydroxy-2-methylpropan-2-yl)-5H-spiro[benzo[d]thiazole-6,4′-piperdin]-4(7H)-one;
rac-(R)-(2-(tert-buty)-4-hydroxy-4,7-dihydro-5H-spiro[benzo[d]thiazole-6,4′-piperdin]-1′-yl)(7-ethoxy-1,3-dimethyl-1H-indazol-5-yl)methanone;
2-(tert-butyl)-1′-(7-ethoxy-3-(hydroxymethyl)-1-methyl-1H-indazole-5-carbonyl)-5H-spiro[benzo[d]thiazole-6,4′-piperdin]-4(7H)-one;
rac-(2-(tert-butyl)-4-hydroxy-4,7-dihydro-5H-spiro[benzo[d]thiazole-6,4′-piperidin]-1′-yl)(7-ethoxy-3-methyl-1H-indazol-5-yl)methanone;
1′-(7-ethoxy-3-methyl-1H-indazole-5-carbonyl)-2-(1-hydroxy-2-methylpropan-2-yl)-5H-spiro[benzo[d]thiazole-6,4′-piperdin]-4(7H)-one;
1′-(7-hydroxy-3-methyl-1H-indazole-5-carbonyl)-2-(1-hydroxy-2-methylpropan-2-yl)-5H-spiro[benzo[d]thiazole-6,4′-piperdin]-4(7H)-one; or, a pharmaceutically acceptable salt thereof, in an isolated form.
   E24. A method of treating acne, comprising administering to the subject a therapeutically effective amount of a compound according to any one of 1 to E16 or a pharmaceutically acceptable salt thereof, or a pharmaceutically acceptable solvate of said compound or salt.
   E25. A method of treating inflammatory skin disease, seborrheic dermatitis, rosacea, steroid acne, papulopustular drug eruption, cutaneous lupus or hidradenitis suppurativa, comprising administering to the subject a therapeutically effective amount of a compound according to any one of E1 to E16 or a pharmaceutically acceptable salt thereof, or a pharmaceutically acceptable solvate of said compound or salt.
   E26. The method according to any of E20 to E25, wherein the compound is administered topically.
   E27. The method according to any of E20 to E25, wherein the compound is administered as a cream, ointment, lotion, gel, solution, suspension, foam, aerosol, spray, shampoo, patch or tape.
   E28. Use of a compound according to any of E1 to E16 for the manufacture of a medicament for the treatment of a disorder for which an ACC inhibitor is indicated.
   E29. Use of a compound according to any of E1 to E16 for the manufacture of a medicament for the treatment of acne.
   E30. A compound according to any of E1 to E16 for use in the treatment of a disorder for which an ACC inhibitor is indicated.

Compounds or compounds in an isolated form that have the same molecular formula but differ in the nature or sequence of bonding of their atoms or the arrangement of their atoms in space are termed “isomers”. Isomers that differ in the arrangement of their atoms in space are termed “stereoisomers”. It will be appreciated by those skilled in the art that the compound or compound in an isolated form of formula I can exist as cis- and frans-achiral diastereomers. An example is the compound or compound in an isolated form of formula IC.

Included within the scope of the described compounds and compounds in an isolated form are all isomers (e.g., cis-, trans-, or diastereomers) of the compounds described herein alone as well as any mixtures. All of these forms, including enantiomers, diastereomers, cis, trans, syn, anti, solvates (including hydrates), tautomers, and mixtures thereof, are included in the described compounds or compounds in an isolated form. Stereoisomeric mixtures, e.g., mixtures of diastereomers, can be separated into their corresponding isomers in a known manner by means of suitable separation methods. Diastereomeric mixtures for example may be separated into their individual diastereomers by means of fractionated crystallization, chromatography, solvent distribution, and similar procedures. This separation may take place either at the level of one of the starting compounds or in a compound of formula I itself. Enantiomers may be separated through the formation of diastereomeric salts, for example by salt formation with an enantiomer-pure chiral acid, or by means of chromatography, for example by HPLC, using chromatographic substrates with chiral ligands.

In therapeutic use for treating disorders in a subject, a compound or compound in an isolated form of the present invention or its pharmaceutical compositions can be administered orally, parenterally, topically, rectally, transmucosally, or intestinally. Parenteral administrations include indirect injections to generate a systemic effect or direct injections to the afflicted area. Topical administrations include the treatment of skin or organs readily accessible by local application, for example, eyes or ears. It also includes transdermal delivery to generate a systemic effect. The rectal administration includes the form of suppositories. The preferred routes of administration are oral and parenteral.

Pharmaceutically acceptable salts of the compounds or compounds in an isolated form of formula I include the acid addition and base salts thereof. Suitable acid addition salts are formed from acids which form non-toxic salts. Examples include the acetate, adipate, aspartate, benzoate, besylate, bicarbonate/carbonate, bisulphate/sulfate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002).

Pharmaceutically acceptable salts of compounds and compounds in an isolated form of formula I may be prepared, respectively, by one or more of three methods: (i) by reacting the compound or compound in an isolated form of formula I with the desired acid or base; (ii) by removing an acid- or base-labile protecting group from a suitable precursor of the compound or compound in an isolated form of formula I, or by ring-opening a suitable cyclic precursor, for example, a lactone or lactam, using the desired acid or base; or (iii) by converting one salt of the compound or compound in an isolated form of formula I to another by reaction with an appropriate acid or base or by means of a suitable ion exchange column. All three reactions are typically carried out in solution. The resulting salt may precipitate out and be collected by filtration or may be recovered by evaporation of the solvent. The degree of ionization in the resulting salt may vary from completely ionized to almost non-ionized.

Pharmaceutical compositions of the present invention may be manufactured by methods well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more pharmaceutically acceptable carriers comprising excipients and auxiliaries, which facilitate processing of the active compound or compound in an isolated form into preparations, which can be used pharmaceutically. Proper formulation is dependent upon the route of administration chosen. Pharmaceutically acceptable excipients and carriers are generally known to those skilled in the art and are thus included in the instant invention. Such excipients and carriers are described, for example, in “Remington's Pharmaceutical Sciences” Mack Pub. Co., New Jersey (1991). The formulations of the invention can be designed to be short-acting, fast-releasing, long-acting, and sustained-releasing. Thus, the pharmaceutical formulations can also be formulated for controlled release or for slow release.

Pharmaceutical compositions suitable for use in the present invention include compositions wherein the active ingredients are contained in an amount sufficient to achieve the intended purpose, i.e., control or the treatment of disorders or diseases. More specifically, a therapeutically effective amount means an amount of compound or compound in an isolated form effective to prevent, alleviate or ameliorate symptoms/signs of disease or prolong the survival of the subject being treated.

The quantity of active component, which is the compound or compound in an isolated form of this invention, in the pharmaceutical composition and unit dosage form thereof, may be varied or adjusted widely depending upon the manner of administration, the potency of the particular compound or compound in an isolated form and the desired concentration.

Determination of a therapeutically effective amount is well within the capability of those skilled in the art. Generally, the quantity of active component will range between 0.01% to 99% by weight of the composition.

Generally, a therapeutically effective amount of dosage of the active component will be in the range of about 0.01 to about 100 mg/kg of body weight/day, preferably about 0.1 to about 10 mg/kg of body weight/day, more preferably about 0.3 to 3 mg/kg of body weight/day, even more preferably about 0.3 to 1.5 mg/kg of body weight/day It is to be understood that the dosages may vary depending upon the requirements of each subject and the severity of the disorders or diseases being treated.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation. If desired, the daily dose may also be divided into multiple doses for administration, e.g., two to four times per day.

Compounds and compounds in an isolated form of the present invention may be administered in a pharmaceutically acceptable form either alone or in combination with one or more additional agents which modulate a mammalian immune system or with anti-inflammatory agents. These agents may include but are not limited to a 5-lipoxygenase activating protein (FLAP) antagonist; a leukotriene antagonist (LTRA) such as an antagonist of LTB4, LTC4, LTD4, LTE4, CysLT1 or CysLT2, e.g., montelukast or zafiriukast; a histamine receptor antagonist, such as a histamine type 1 receptor antagonist or a histamine type 2 receptor antagonist, e.g., loratidine, fexofenadine, desloratidine, levocetirizine, methapyrilene or cetirizine; an α1-adrenoceptor agonist or an α2-adrenoceptor agonist, e.g., phenylephrine, methoxamine, oxymetazoline or methylnorephrine; a muscarinic M3 receptor antagonist, e.g. tiotropium or ipratropium; a dual muscarinic M3 receptor antagononist/β2 agonist; a PDE inhibitor, such as a PDE3 inhibitor, a PDE4 inhibitor or a PDE5 inhibitor, e.g., theophylline, sildenafil, vardenafil, tadalafil, ibudilast, cilomilast or roflumilast; sodium cromoglycate or sodium nedocromil; a cyclooxygenase (COX) inhibitor, such as a non-selective inhibitor (e.g., aspirin or ibuprofen) or a selective inhibitor (e.g. celecoxib or valdecoxib); a glucocorticosteroid, e.g., fluticasone, mometasone, dexamethasone, prednisolone, budesonide, ciclesonide or beclamethasone; an anti-inflammatory monoclonal antibody, e.g., infliximab, adalimumab, tanezumab, ranibizumab, bevacizumab or mepolizumab; a s2 agonist, e.g., salmeterol, albuterol, salbutamol, fenoterol or formoterol, particularly a long-acting s2 agonist; an integrin antagonist, e.g., natalizumab; an adhesion molecule inhibitor, such as a VLA-4 antagonist; a kinin B1 or B2 receptor antagonist; an immunosuppressive agent, such as an inhibitor of the IgE pathway (e.g., omalizumab) or cyclosporine; a matrix metalloprotease (MMP) inhibitor, such as an inhibitor of MMP-9 or MMP-12; a tachykinin NK1, NK2 or NK3 receptor antagonist; a protease inhibitor, such as an inhibitor of elastase, chymase or catheopsin G; an adenosine A2a receptor agonist; an adenosine A2b receptor antagonist; a urokinase inhibitor; a dopamine receptor agonist (e.g., ropinirole), particularly a dopamine D2 receptor agonist (e.g., bromocriptine); a modulator of the NFκB pathway, such as an IKK inhibitor; a further modulator of a cytokine signaling pathway such as an inhibitor of JAK kinase, syk kinase, p38 kinase, SPHK-1 kinase, Rho kinase, EGF-R or MK-2; a mucolytic, mucokinetic or anti-tussive agent; an antibiotic; an antiviral agent; a vaccine; a chemokine; an epithelial sodium channel (ENaC) blocker or Epithelial sodium channel (ENaC) inhibitor; a nucleotide receptor agonist, such as a P2Y2 agonist; a thromboxane inhibitor; niacin; a 5-lipoxygenase (5-LO) inhibitor, e.g., Zileuton; an adhesion factor, such as VLAM, ICAM or ELAM; a CRTH2 receptor (DP2) antagonist; a prostaglandin D2 receptor (DP1) antagonist; a haematopoietic prostaglandin D2 synthase (HPGDS) inhibitor; interferon-β; a soluble human TNF receptor, e.g., Etanercept; a HDAC inhibitor; a phosphoinositotide 3-kinase gamma (P13Kγ) inhibitor; a phosphoinositide 3-kinase delta (P13Kδ) inhibitor; a CXCR-1 or a CXCR-2 receptor antagonist; an IRAK-4 inhibitor; and, a TLR-4 or TLR-9 inhibitor, including the pharmaceutically acceptable salts of the specifically named compounds. The agents may be administered with another active agent, wherein the second active agent may be administered either orally or topically.

Suitable specific agents for use in combination therapy with a compound or compound in an isolated form of formula 1, or a pharmaceutically acceptable salt thereof, sulfasalazine, mesalazine, prednisone, azathioprine, infliximab, adalimumab, belimumab, becertolizumab, natalizumab, vedolizumab, hydrocortisone, budesonide, cyclosporin, tacrolimus, fexofenadine, 6-mercaptopurine, methotrexate, ursodeoxycholic acid, obeticholic acid, anti-histamines, rifampin, prednisone, methotrexate, azathioprine, cyclophosphamide, hydroxychloroquine, mofetil, sodium mycophenolate, tacrolimus, leflunomide, chloroquine and quinacrine, thalidomide, rituxan, NSAIDs, solumedrol, depomedrol and dexamethasone.

### Chemical Synthesis

In executing the synthesis of the compounds of the invention, one skilled in the art will recognize the need to sample and assay reaction mixtures prior to work up in order to monitor the progress of reactions and decide whether the reaction should be continued or whether it is ready to be worked up to obtain the desired product. Common methods for assaying reaction mixtures include thin-layer chromatography (TLC), liquid chromatography/mass spectroscopy (LCMS), and nuclear magnetic resonance (NMR).

One skilled in the art will also recognize that the compounds and compounds in an isolated form of the invention may be prepared as mixtures of diastereomers or geometric isomers (e.g., cis and trans substitution on a cycloalkane ring). These isomers can be separated by standard chromatographic techniques, such as normal phase chromatography on silica gel, reverse phase preparative high pressure liquid chromatography or supercritical fluid chromatography. One skilled in the art will also recognize that some compounds of the invention are chiral and thus may be prepared as racemic or scalemic mixtures of enantiomers. Several methods are available and are well known to those skilled in the art for the separation of enantiomers. A preferred method for the routine separation enantiomers is supercritical fluid chromatography employing a chiral stationary phase.

### EXPERIMENTAL SECTION

Except where otherwise noted, reactions were run under an atmosphere of nitrogen. Chromatography on silica gel was carried out using 250-400 mesh silica gel using pressurized nitrogen (˜10-15 psi) to drive solvent through the column (“flash chromatography”). Where indicated, solutions and reaction mixtures were concentrated by rotary evaporation under vacuum.

### Preparations and Examples

### Preparation 1: 2-(tert-butyl)-5H-spiro[benzo[d]thiazole-6.4′-piperdin]-4(7H)-one hydrochloride salt (P1)

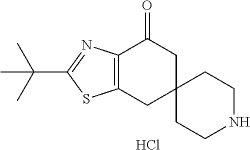 [SMILES: CC(C)(C)c1nc2c(s1)CC1(CCNCC1)CC2=O ; Cl]

### Step 1: Synthesis of tert-butyl 2-(tert-butyl)-7H-spiro[benzo[d]thiazole-6.4′-piperidine]-1′-carboxylate (C1)

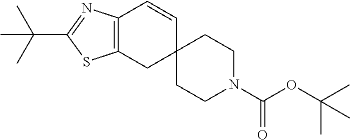 [SMILES: CC(C)(C)OC(=O)N1CCC2(C=Cc3nc(C(C)(C)C)sc3C2)CC1]

To a solution of tert-butyl 9-oxo-3-azaspiro[5.5]undec-7-ene-3-carboxylate (CAS: 873924-07-3; 160 g, 603 mmol) in DCM (2.4 L) was added Et₃N (251 mL, 1.81 mol). The solution was cooled to about −78° C. and a solution of TMSOTf (164 mL, 904 mmol) in DCM (800 mL) was added dropwise. The solution was stirred at about −78° C. for about 30 min, then warmed to about 0° C. and stirred for about 2 h at about 0° C. The solution was cooled to about −78° C. and a suspension of NBS (118 g, 663 mmol) in DCM (800 mL) was added. The mixture was stirred at about −78° C. for about 2 h, then warmed to about 0° C. Boc₂O (52.6 g, 241 mmol) was added, stirring the mixture at about 0° C. for about 2 h. Saturated NaHCO₃ (aq) (1.6 L) was added, and the organic phase was separated and concentrated under reduced pressure. To the residue was added 2,2-dimethylpropanethioamide (84.8 g, 723 mmol), pyridine (436 mL, 542 mmol, and EtOH (2.1 L). The solution was heated to about 80° C. for about 16 h and then cooled to about 23° C. The mixture was washed with saturated NaHCO₃ (aq) (2.0 L) and the organic phase was concentrated under reduced pressure. The residue was purified by column chromatography (silica, petroleum ether EtOAc=1: 0 to 3: 1) to provide the title compound (149 g, 68%). ¹H NMR (400 MHz, CDCl₃) δ=6.65 (d, 1H), 5.87 (d, 1H), 3.57-3.51 (m, 2H), 3.40-3.33 (m, 2H), 2.82 (s, 2H), 1.71-1.67 (m, 2H), 1.53-1.48 (m, 2H), 1.46 (s, 9H), 1.43 (s, 9H); LC/MS m/z (M+H)⁺=363.1.

Step 2: Synthesis of 2-(tert-butyl-5H-siro[benzo[d]thiazole-6.4′-piperidin]-4(7H)-one hydrochloride (P1)

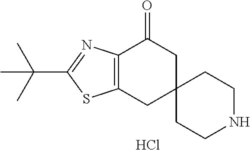 [SMILES: CC(C)(C)c1nc2c(s1)CC1(CCNCC1)CC2=O ; Cl]

To compound C1 (188 g, 519 mmol) was added MeOH (1.88 L). The solution was cooled to about 15° C. and NBS (96.9 g, 545 mmol) was added in batches. The resulting mixture was stirred at about 15° C. for about 2 h and concentrated under reduced pressure. To the residue was added THF (2.5 L) and the resulting solution was cooled to about 15° C. KOtBu (1 M in THF, 2.1 L) was added and the solution was stirred at about 15° C. for 16 h. Water (4.9 L) was added and the mixture was extracted with isopropyl acetate (3 X 2.8 L). The combined organic phases were concentrated under reduced pressure to provide crude tert-butyl 2-(tert-butyl)-4-methoxy-7H-spiro[benzo[d]thiazole-6,4′-piperidine]-1′-carboxylate (intermediate C1b). To the resulting residue was added additional intermediate C1b (21.6 g, 55 mmol) from a prior batch and dioxane (970 mL), followed by dropwise addition of HCl (4 M in dioxane, 2.26 L). The solution was stirred at about 25° C. for about 16 h and then concentrated under reduced pressure. MeOH (3.5 L) was added and the solution was heated to about 60° C. and cooled to about 25° C. The solution was concentrated under reduced pressure to remove 2.8 L of MeOH.

The resulting mixture was stirred at about 25° C. for about 16 h. EtOAc (2.0 L) was added, and the resulting mixture was stirred at about 25° C. for about 16 h. The resulting slurry was filtered, and the cake was dried under reduced pressure at about 40° C. to afford the title compound (157 g, 87%). ¹H NMR (400 MHz, DMSO-d6) δ=9.01 (br s, 1H), 8.93 (br s, 1H), 3.22 (s, 2H), 3.06 (br s, 4H), 2.62 (s, 2H), 1.76-1.65 (m, 4H), 1.37 (s, 9H); LC/MS m/z (M+H)⁺=279.2.

### Example 1: 2-(tert-butyl)-1′-(7-ethoxy-3-methyl-1H-Indazole-5-carbonyl)-5H-spiro[benzo[d]thiazole-6,4′-piperidin]-4(7H)-one (M1 metabolite)

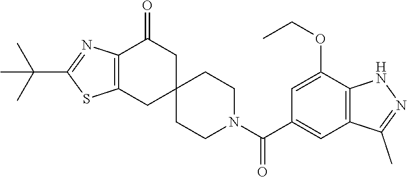 [SMILES: CCOc1cc(C(=O)N2CCC3(CC2)CC(=O)c2nc(C(C)(C)C)sc2C3)cc2c(C)n[nH]c12]

### Step 1: Synthesis of ethyl 7-ethoxy-3-methyl-1H-indazole-5-carboxylate (A1)

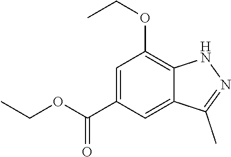 [SMILES: CCOC(=O)c1cc(OCC)c2[nH]nc(C)c2c1]

To a solution of ethyl 7-hydroxy-3-methyl-1H-indazole-5-carboxylate (0.11 g, 0.5 mmol; prepared as described by PCT patent publication WO 2009/144554) in toluene (2.5 mL) was added Tsunoda reagent (0.17 g, 0.7 mmol) and ethanol (29.2 μL, 0.5 mmol). The reaction was heated at about 100° C. for about 16 h and then concentrated. The residue was diluted with water (25 mL) and extracted with EtOAc (2×25 mL). The combined EtOAc extracts were washed with sat. Na₂CO₃ (2×25 mL), brine (2×25 mL), dried over MgSO₄, filtered, and concentrated. The residue was purified by chromatography (silica, EtOAc/Hexanes, 0-50%) to provide the title compound (0.075 g, 60% yield) as a white solid. ¹H NMR (500 MHz, Methanol-d4) δ 8.04 (s, 1H), 7.36 (s, 1H), 4.38 (q, 2H, 18), 4.26 (q, 2H), 2.56 (s, 2H), 1.52 (t, 3H), 1.41 (t, 3H); 13C NMR (126 MHz, Methanol-d4) δ 168.6, 145.8, 145.5, 136.7, 124.8, 124.6, 117.0, 106.6, 65.2, 62.1, 15.0, 14.7, 11.7; LC/MS m/z (M+H)⁺=249.2.

Step 2: Synthesis of 7-ethoxy-3-methyl-1H-indazole-5-carboxylic acid (A2)

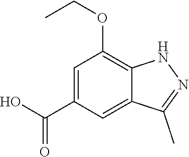 [SMILES: CCOc1cc(C(=O)O)cc2c(C)n[nH]c12]

To compound A1 (0.12 g, 0.48 mmol) in EtOH (2.0 mL) and THF (2.0 mL) was added 1 M aqueous NaOH (2.0 mL, 4.0 mmol. The mixture was stirred at about 25° C. for about 17 h, then concentrated under reduced pressure. 1.5 M aqueous citric acid (2.5 mL) and water (2 mL) were added to the mixture. After stirring for about 3 minutes, the precipitate was isolated via filtration to provide the title compound (0.08 mg, 75%). ¹H NMR (400 MHz, DMSO-d6) δ 12.46 (s, 1H), 7.97 (d, 1H), 7.28 (s, 1H), 4.26 (q, 2H), 2.55 (s, 3H), 1.44 (t, 3H).

### Step 3: 2-(tert-butyl)-1′-(7-ethoxy-3-methyl-1H-indazole-5-carbonyl)-5H-spiro[benzo[d]thiazole-6.4′-piperdin]-4(7H)-one

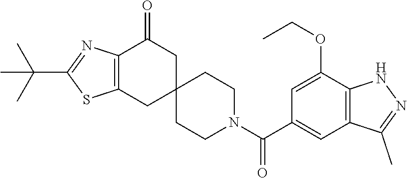 [SMILES: CCOc1cc(C(=O)N2CCC3(CC2)CC(=O)c2nc(C(C)(C)C)sc2C3)cc2c(C)n[nH]c12]

To a solution of preparation P1 (0.279 g, 1.82 mmol, A2 (0.4 g, 1.82 mmol, EDCl·HCl (0.453 g, 2.36 mmol) and HOBt (0.319 g, 2.36 mmol) in DMF (18.2 mL) at about 25° C. was added Et₃N (1.27 mL, 9.08 mmol. The mixture was stirred at about 25° C. for about 16 h, then diluted with EtOAc (50 mL). The mixture was washed with saturated aqueous Na₂CO₃ (2×25 mL) and brine (2×25 mL). The organic phase was dried over MgSO₄, filtered, and concentrated. The residue was purified by chromatography (silica, DCM: MeOH, 0-20%) to provide the title compound (0.711 g, 81.4%) as a white solid. ¹H NMR (400 MHz, Methanol-d4) δ 7.38 (d, 1H), 6.85 (s, 1H), 4.29 (q, 2H), 3.77 (m, 4H), 3.28 (s, 2H), 2.76 (s, 2H), 2.56 (s, 3H), 1.72 (m, 4H), 1.54 (t, 3H), 1.46 (s, 9H); LC/MS m/z (M+H)⁺=481.4.

### Example 2: 2-(tert-butyl)-1′-(7-hydroxy-1,3-dimethyl-1H-Indazole-5-carbonyl)-5H-spiro[benzo[d]thiazole-6,4′piperidin]-7H)-one (M2 metabolite)

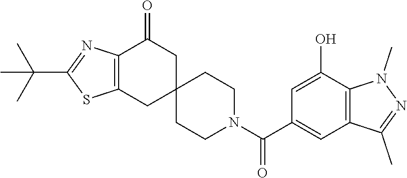 [SMILES: Cc1nn(C)c2c(O)cc(C(=O)N3CCC4(CC3)CC(=O)c3nc(C(C)(C)C)sc3C4)cc12]

To a solution of preparation P1 (1.39 g, 3.0 mmol), ethyl 7-hydroxy-1,3-dimethyl-1H-indazole-5-carboxylate (as describe in U.S. Ser. No. 63/393,712) (0.7 g, 3.43 mmol, EDCl·HCl (0.69 g, 3.6 mmol) and HOBt (0.57 g, 3.67 mmol) in DMF (15 mL) at about 25° C. was added DIEA (3.2 mL, 18.0 mmol). The mixture was stirred at about 25° C. for about 16 h, then diluted with EtOAc (50 mL). The mixture was washed with 10% aqueous citric acid (25 mL), saturated aqueous NaHCO₃ (50 mL), and brine (30 mL). The organic phase was dried over MgSO₄, filtered, and concentrated. The residue was purified by chromatography (silica, DCM: MeOH, 0-10%) to provide the title compound (0.71 g, 50%) as a white solid. ¹H NMR (400 MHz, DMSO-d6) δ 10.35 (s, 1H), 7.15 (d, 1H), 6.68 (d, 1H), 4.15 (s, 3H), 3.51 (m, 4H), 3.23 (s, 2H), 2.65 (s, 2H), 2.42 (s, 3H), 1.55 (m, 4H), 1.39 (s, 9H); LC/MS m/z (M+H)⁺=467.4

### Example 3: 1′-(7-ethoxy-1,3-dimethyl-1H-Indazole-5-carbonyl)-2-(1-hydroxy-2-methylpropan-2-yl)-5H-spiro[benzo[d]thiazole-6,4′-piperidin]-4(7H)-one M3 Metabolite

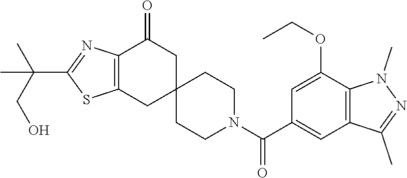 [SMILES: CCOc1cc(C(=O)N2CCC3(CC2)CC(=O)c2nc(C(C)(C)CO)sc2C3)cc2c(C)nn(C)c12]

### Step 1: Synthesis of 3-((tert-butyldimethylsilyl)oxy)-2.2-dimethylpropanamide (A3)

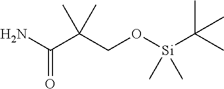

To a solution of 3-((tert-butyldimethylsilyl)oxy)-2,2-dimethylpropanoic acid (8 g, 34.43 mmol; prepared as described by WO 2006/114774) in THF (80 mL) was added DIEA (13.35 g, 103.27 mmol) and isobutyl chloroformate (9.4 g, 68.85 mmol) at 0° C. The mixture was stirred for about i h at about 0° C. NH3·H2O (12.06 g, 103.27 mmol) was added to the mixture at about 0° C. The mixture was stirred for an additional 1 h at about 0° C. The mixture was diluted with water (100 mL) and extracted with ethyl acetate (3×80 mL). The organic phase was washed with brine (100 mL), dried over Na₂SO₄, filtered, and concentrated under reduced pressure. The residue was purified by chromatography (silica, EtOAc: Pet. Ether, 0-10%) to provide the title compound (6.5 g, 81.5%) as a white solid. ¹H NMR (400 MHz, Chloroform-d) δ 5.38 (s, 1H), 4.63 (s, 1H), 3.45 (s, 2H), 1.05 (s, 6H), 0.87-0.78 (m, 9H), 0.00 (s, 6H).

### Step 2: Synthesis of 3-hydroxy-2.2-dimethylpropanamide (A4)

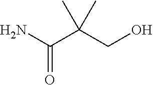

A solution of A3 (6.1 g, 26.4 mmol) was dissolved in HCl/dioxane (4 N, 60 ml) at 20° C. The reaction was stirred for about 1 h at about 20° C. The mixture was concentrated under reduced pressure to provide the title compound (7 g, 100%) as a brown solid; LC/MS m/z (M+H)⁺=118.1

### Step 3: Synthesis of 3-((tert-butyldiohenylsiyl)oxy)-2.2-dimethylpropanamide (A5)

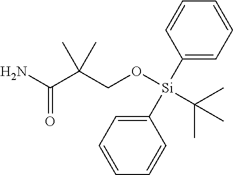 [SMILES: CC(C)(CO[Si](c1ccccc1)(c1ccccc1)C(C)(C)C)C(N)=O]

To a solution of A4 (7 g, 25.61 mmol) in DMF (70 mL) was added imidazole (26.2 g, 38.41 mmol) and tert-butylchlorodiphenylsilane (8.45 g, 30.73 mmol) at about 20° C. The reaction was stirred for about 12 h at about 20° C. The reaction was diluted with water (50 mL) and extracted with ethyl acetate (3×50 mL). The organic phase was washed with brine (50 mL), dried over Na₂SO₄, filtered, and concentrated under reduced pressure. The residue was purified by chromatography (silica, EtOAc: Pet. Ether, 0-5%) to provide the title compound (7.3 g, 80%) as a white solid; ¹H NMR (400 MHz, Chloroform-d) δ 7.56 (m, 4H), 7.40-7.26 (m, 6H), 5.23 (s, 1H), 4.48 (s, 1H), 3.50 (s, 2H), 1.05 (s, 6H), 0.99 (s, 9H).

### Step 4: Synthesis of 3-((tert-butyldiphenylsilyl)oxy)-2.2-dimethylpropanethioamide (A6)

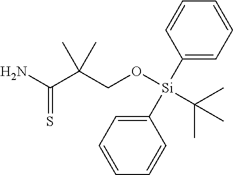 [SMILES: CC(C)(CO[Si](c1ccccc1)(c1ccccc1)C(C)(C)C)C(N)=S]

To a solution of A5 (7.3 g, 20.53 mmol) in DCM (70 mL) was added Lawesson's reagent (9.97, 24.64 mmol) at about 20° C. The reaction was stirred for about 12 h at about 20° C. The reaction was diluted with water (50 mL) and extracted with DCM (3×50 mL). The organic phase was dried over Na₂SO₄, filtered, and concentrated under reduced pressure. The residue was purified by chromatography (silica, EtOAc: Pet. Ether, 0-5%) to provide the title compound (4.7 g, 58.9%) as a white solid; ¹H NMR (400 MHz, Chloroform-d) δ 8.40 (s, 2H), 7.58-7.51 (m, 4H), 7.41-7.26 (m, 6H), 3.60 (s, 2H), 1.19 (s, 6H), 0.98 (s, 9H).

### Step 5: Synthesis of tert-butyl 10-bromo-9-oxo-3-azaspiro[5.5]undec-7-ene-3-carboxylate (A7)

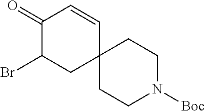 [SMILES: CC(C)(C)OC(=O)N1CCC2(C=CC(=O)C(Br)C2)CC1]

A solution of tert-butyl 9-oxo-3-azaspiro[5.5]undec-7-ene-3-carboxylate (CAS: 873924-07-3 (6 g, 22.62 mmol) in THF (60 mL) was added PhMe₃NBr₃ (9.78 g, 26.0 mmol) at 0° C. The reaction was stirred for about 12 h at about 20° C. in the dark shade. The reaction was poured into saturated NaHCO₃ (60 mL) and extracted with ethyl acetate (3×60 mL). The organic phase was washed with brine (100 mL), dried over Na₂SO₄, filtered, and concentrated under reduced pressure. The residue was purified by chromatography (silica, EtOAc: Pet. Ether, 0-30%) to provide the title compound (4.6 g, 59.1%) as a white solid; ¹H NMR (400 MHz, Chloroform-d) δ 6.90 (dd, 1H), 6.12 (d, 1H), 4.83 (dd, 1H), 3.68-3.54 (m, 1H), 3.54-3.39 (m, 3H), 2.76-2.65 (m, 2H), 2.12-1.95 (m, 2H), 1.83-1.58 (m, 2H), 1.51 (s, 9H).

### Step 6: Synthesis of tert-butyl 2-(1-((tert-butyldiphenylsilyl)oxy)-2-methylpropan-2-yl-7H-spiro[benzo[d]thiazole-6.4′-piperidine]-1′-carboxylate(A8)

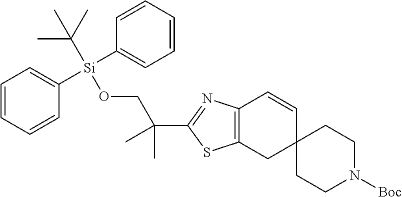 [SMILES: CC(C)(C)OC(=O)N1CCC2(C=Cc3nc(C(C)(C)CO[Si](c4ccccc4)(c4ccccc4)C(C)(C)C)sc3C2)CC1]

To a solution of A7 (4.6 g, 13.45 mmol) in EtOH (50 mL) was added compound A6 (2 g, 5.38 mmol) and pyridine (3.83 g, 48.44 mmol) at about 20° C. The reaction was stirred for about 48 h at about 80° C. The reaction was concentrated under reduced pressure. The residue was diluted with water (50 mL) and extracted with ethyl acetate (3×50 mL). The organic phase was dried over Na₂SO₄, filtered, and concentrated under reduced pressure. The residue was purified by chromatography (silica, EtOAc: Pet. Ether, 0-5%) to provide the title compound (1.9 g, 57.2%) as a white solid; ¹H NMR (400 MHz, Chloroform-d) δ 7.49 (dt, 4H), 7.39-7.28 (m, 2H), 7.28 (dd, 4H), 6.59 (d, 1H), 5.80 (d, 1H), 3.64 (s, 2H), 3.45 (d, 2H), 3.29 (ddd, 2H), 2.76 (s, 2H), 1.65-1.55 (m, 2H), 1.48 (s, 2H), 1.39 (s, 9H), 1.37 (s, 6H), 0.93 (s, 9H).

### Step 7: Synthesis of tert-butyl 5-bromo-2-(1-((tert-butyldiphenylsilyl)oxy)-2-methylpropan-2-yl)-4-methoxy-4.7-dihydro-5H-spiro[benzo[d]thiazole-6.4′-piperidine]-1′-carboxylate (A9)

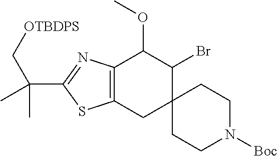

To a solution of compound A8 (1.9 g, 3.08 mmol) in MeOH (20 mL) was added NBS (657 mg, 3.7 mmol) at about 20° C. The reaction was stirred for 1 h at 20° C. The reaction was concentrated under reduced pressure. The residue was diluted with water (20 mL) and extracted with EtOAc (3×20 mL). The organic phase was dried over Na₂SO₄, filtered, and concentrated under reduced pressure. The residue was purified by chromatography (silica, EtOAc: Pet. Ether, 0-10%) to provide the title compound A9 (1.4 g, 62.5%) as a white solid. ¹H NMR (400 MHz, CDCl₃) δ 7.61 (ddt, 4H), 7.49-7.33 (m, 6H), 4.79 (d, 1H), 4.47 (d, 1H), 3.68-3.52 (m, 7H), 3.33-3.13 (m, 2H), 2.86 (s, 2H), 1.99-1.88 (m, 2H), 1.77 (dt, 2H), 1.47 (s, 15H), 1.02 (d, 1H), 1.01 (s, 9H).

Step 8: Synthesis of tert-butyl 2-(1-((tert-butyldiphenylsilyl)oxy)-2-methylpropan-2-yl)-4-oxo-4,7-dihydro-5H-spiro[benzo[d]thiazole-6,4′-piperidine]-1′-carboxylate (A10) and tert-butyl 2-(1-hydroxy-2-methylpropan-2-yl)-4-oxo-4,7-dihydro-5H-spiro[benzo[d]thiazole-6,4′-piperidine]-1′-carboxylate (A11)

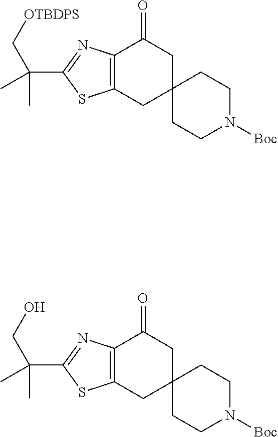

To a solution of compound A9 (1.4 g, 1.92 mmol) in THF (15 mL) was added t-BuOK (3.85 mL, 1N, 3.85 mmol) at 0° C. The reaction was stirred for 1 h at 20° C. Then the reaction was adjusted pH to 2 with HCl (1N) at 0° C. The reaction was stirred for 1 h at 0° C. The reaction was concentrated under reduced pressure. The residue was diluted with water (20 mL) and extracted with EtOAc (3×20 mL). The organic phase was dried over Na₂SO₄, filtered, and concentrated under reduced pressure. The residue was purified by chromatography (silica, EtOAc: Pet. Ether, 0-50%) to provide the title compound A10 (690 mg, 56.5%) as a white solid; ¹H NMR (400 MHz, CDCl₃) δ 7.58 (dt, 4H), 7.48-7.31 (m, 6H), 3.75 (s, 2H), 3.50-3.36 (m, 4H), 3.05 (s, 2H), 2.65 (s, 2H), 1.58 (q, 4H), 1.47 (s, 15H), 1.01 (s, 9H) and All (220 mg, 29.0%) as a brown solid; 1H NMR (400 MHz, CDCl3) δ 4.16 (t, 1H), 3.81 (d, 2H), 3.60-3.38 (m, 4H), 3.09 (s, 2H), 2.68 (s, 2H), 1.64 (m, 4H), 1.48 (d, 15H).

### Step 9: Synthesis of 2-(1-hydroxy-2-methylpropan-2-yl)-5H-spiro[benzo[d]thiazole-6,4′-piperidin]-4 (7H)-one hydrochloride (A12)

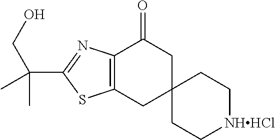

A mixture of A10 (690 mg, 56.5%) and All (220 mg, 0.557 mmol) was dissolved in HCl/dioxane (4N, 2 mL) at 20° C. The reaction was stirred for 1 h at 20° C. The reaction was concentrated under reduced pressure to give A12 (200 mg, 39%) as a white solid; 1H NMR (400 MHz, MeOD) δ 3.70 (s, 2H), 3.35-3.19 (m, 6H), 2.77 (s, 2H), 1.89 (m, 4H), 1.43 (s, 6H).

### Step 10: Synthesis of 1′-(7-ethoxy-1,3-dimethyl-1H-indazole-5-carbonyl)-2-(1-hydroxy-2-methylpropan-2-yl)-5H-spiro[benzo[d]thiazole-6,4′-piperidin]-4 (7H)-one

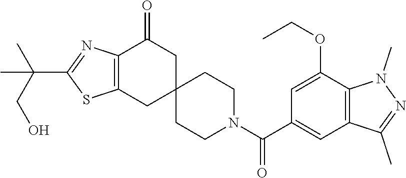 [SMILES: CCOc1cc(C(=O)N2CCC3(CC2)CC(=O)c2nc(C(C)(C)CO)sc2C3)cc2c(C)nn(C)c12]

To a solution of A12 (50 mg, 169.83 μmmol) in MeCN (0.5 mL) and DMF (0.5 mL) was added 7-ethoxy-1,3-dimethyl-1H-indazole-5-carboxylic acid C11 (39.7 mg, 169.83 μmmol), TCFH (95.3 mg, 339.66 μmmol) and NMI (69.7 mg, 849.15 μmmol) at 20° C. The reaction was stirred for 12 h at 20° C. The reaction was purified with pre-HPLC and lyophilized to give the title compound (25.1 mg, 28.9%) as a white solid; ¹H NMR (400 MHz, MeOD) δ 7.33 (s, 1H), 6.84 (s, 1H), 4.31-4.20 (m, 5H), 3.69 (m, 6H), 3.27 (s, 2H), 2.75 (s, 2H), 2.51 (s, 3H), 1.71 (m, 4H), 1.54 (t, 3H), 1.41 (s, 6H); LC/MS m/z (M+H)⁺=511.1 Example 4: rac-(R)H2-(tert-butyl)-4-hydroxy-4,7-dihydro-5H-spiro[benzo[d]thiazole-6,4′-piperidin]-1′-yl)(7-ethoxy-1,3-dimethyl-1H-Indazol-5-yl)methanone M4 Metabolite rac

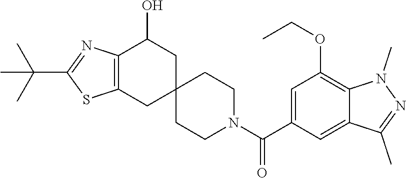 [SMILES: CCOc1cc(C(=O)N2CCC3(CC2)Cc2sc(C(C)(C)C)nc2C(O)C3)cc2c(C)nn(C)c12]

To a solution of 2-(tert-butyl)-1-(7-ethoxy-1,3-dimethyl-1H-indazole-5-carbonyl)-5H-spiro[benzo[d]thiazole-6,4′-piperdin]-4(7H)-one (Example 14) (100 mg, 0.2 mmol) in MeOH (3.5 mL) was added NaBH₄ (23 mg, 0.6 mmol) and stirred at about 25° C. for about 17 h. The reaction was diluted with 10% MeOH-EtOAc (20 mL) and water (20 mL) and organic layer was separated. The aqueous layer was extracted using EtOAc (2×20 mL). The combined EtOAc extracts were dried over MgSO₄, filtered, and concentrated under reduced pressure. The residue was purified by HPLC (Column: Xbidge™ C18, 19×100 mm, 5 μm) Mobile phase A-0.05% TFA in water (v/v); Mobile phase B: 0.05% TFA in acetonitrile (v/v), 5-95 B gradient time of 10 min to provide the title compound (67 mg, 68%); ¹H NMR (600 MHz, DMSO-d6) δ 7.27 (s, 1H), 6.77 (s, 1H), 5.35-5.02 (m, 1H), 4.62 (t, 1H), 4.19 (q, 2H), 4.14 (s, 3H), 3.45-3.39 (m, 4H, overlap with d-DMSO), 2.75 (d, 1H), 2.68 (d, 1H), 2.43 (s, 3H), 1.99 (dd, 1H), 1.72 (dd, 1H), 1.69-1.59 (m, 2H), 1.53 (dd, 2H), 1.44 (t, 3H), 1.35 (s, 9H); LC/MS m/z (M+H)⁺=497.3.

### Example 4a and 4b: (R)-(2-(tert-butyl)-4-hydroxy-4,7-dihydro-5H-spiro[benzo[d]thiazole-6,4′-piperidin]-1′-yl)(7-ethoxy-1,3-dimethyl-1H-indazol-5-yl)methanone and (S)2-(tert-butyl)-4-hydroxy-4,7-dihydro-5H-spiro[benzo[d]thiazole-6,4′-piperidin]-1′-yl)(7-ethoxy-1,3-dimethyl-1H-Indazol-5-yl)methanone

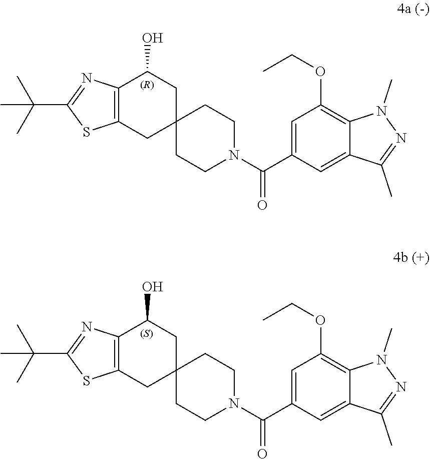

### Example 4 (3.6 g) rac-(R)-(2-(tert-butyl)-4-hydroxy-4,7-dihydro-5H-spiro[benzo[d]thiazole-6,4′-piperidin]-1′-yl)(7-ethoxy-1,3-dimethyl-1H-indazol-5-yl)methanone was purified by chiral SFC (Chiralcel™ OZ-H 250 mm×4.6 mm×5 μm column with a mobile phase of CO_{2(g)}/MeOH: Acetonitrile (1:1)=60:40 and a flow rate of 80 mL/min) to provide

4 a: (1.48 g, 40.9%) as a white solid, retention time=5.71 min, 100% ee; [α]_{D}²⁰=−9.33 (c=1.5, MeOH); LC/MS m/z (M+H)⁺=497.6. Absolute stereochemistry was determined by mosher ester analysis of R-MTPA and S-MTPA ester of 4a. (MTPA: α-methoxy-α-trifluoromethyl phenylacetic Acid)

4 b: (1.48 g, 40.9%) as a white solid, retention time=6.35 min, 100% ee; [α]_{D}²⁰=+10.8 (c=1.5, MeOH); LC/MS m/z (M+H)⁺=497.6. Absolute stereochemistry determined by mosher ester analysis of R-MTPA and S-MTPA ester of 4b.

¹H NMR (400 MHz, DMSO-d₆) δ 7.28 (s, 1H), 6.79 (s, 1H), 5.16 (d, 1H), 4.63 (d, 1H), 4.22 (t, 2H), 4.15 (s, 3H), 3.74-3.39 (m, 4H), 2.72 (q, 2H), 2.44 (s, 3H), 2.00 (dd, 1H), 1.79-1.50 (m, 5H), 1.45 (t, 3H), 1.36 (s, 9H).

### Example 5: 2-(tert-butyl)-1′-(7-ethoxy-3-(hydroxymethyl)-1-methyl-1H-Indazole-5-carbonyl)-spiro[benzo[d]thiazole-6,4′piperidn]-4(7H)-one M5 Metabolite

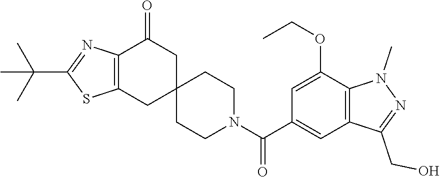 [SMILES: CCOc1cc(C(=O)N2CCC3(CC2)CC(=O)c2nc(C(C)(C)C)sc2C3)cc2c(CO)nn(C)c12]

To a solution of P1 (225 mg, 0.71 mmol), 7-ethoxy-3-(hydroxymethyl)-1-methyl-1H-indazole-5-carboxylic acid (170 mg, 0.68 mmol) and EDCl·HCl (260 mg, 1.36 mmol) in pyridine (5.0 mL) was heated in microwave at 110° C. for 30 min. The solvent was concentrated, and the residue was purified by prep-HPLC (Column: C18, 30×150 mm, 5 μm; Mobile Phase A 0.05% NH₄0H in water (v/v); Mobile phase B: 0.05% NH40H in MeCN (v/v); 22-52% B gradient, 9 min, hold at 100% B for 2 min; flow rate 30 mL/min, to provide the title compound (133.1 mg, 38.4%). LC/MS m/z (M+Na)=533.4; ¹H NMR (400 MHz, MeOD) δ 7.49 (d, 1H), 6.85 (d, 1H), 4.89 (s, 2H), 4.27 (s, 3H), 4.26 (q, 2H), 3.80 (s, 2H), 3.60 (s, 2H), 3.27 (s, 2H), 2.75 (s, 2H), 1.75 (s, 2H), 1.67 (s, 2H), 1.55 (t, 3H), 1.45 (s, 9H).

### Example 6: rac-(2-(tert-butyl)-4-hydroxy-4,7-dihydro-5H-spiro[benzo[d]thiazole-6,4′-piperidin]-1′-yl)(7-ethoxy-3-methyl-1H-Indazol-5-yl)methanone M6 Metabolite

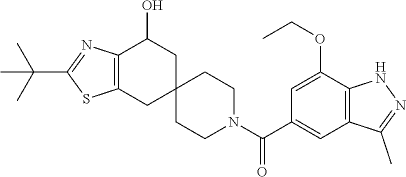 [SMILES: CCOc1cc(C(=O)N2CCC3(CC2)Cc2sc(C(C)(C)C)nc2C(O)C3)cc2c(C)n[nH]c12]

Prepared in the same manner as Example 4, using 2-(tert-butyl)-1′-(7-ethoxy-3-methyl-1H-indazole-5-carbonyl)-5H-spiro[benzo[d]thiazole-6,4′-piperidin]-4(7H)-one (288 mg, 0.6 mmol) to give title compound (252 mg, 87.1%) as a white solid; LC/MS m/z (M+H)⁺=483.1; ¹H NMR (600 MHz, DMSO) δ 12.96 (s, 1H), 7.29 (s, 1H), 6.78 (s, 1H), 5.17 (dd, 1H), 4.62 (d, 1H), 4.23 (q, 2H), 3.44 (m, 4H), 2.83-2.63 (m, 2H), 2.55 (s, 3H), 2.46 (s, 2H), 1.99 (dd, 2H), 1.72 (dd, 2H), 1.43 (t, 3H), 1.35 (s, 9H).

### Example 7: 1′-(7-ethoxy-3-methyl-1H-Indazole-5-carbonyl)-2-(1-hydroxy-2-methylpropan-2-yl)-5H-spiro[benzo[d]thiazole-6,4′-piperidin]-4(7H)-one M7 Metabolite

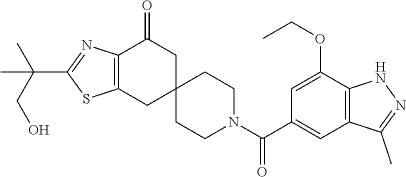 [SMILES: CCOc1cc(C(=O)N2CCC3(CC2)CC(=O)c2nc(C(C)(C)CO)sc2C3)cc2c(C)n[nH]c12]

To a solution of A12 (120 mg, 0.36 mmol, A2 (80 mg, 0.36 mmol) in MeCN (4.0 mL) was added TCFH (0.153 mg, 0.55 mmol) and NMI (89.5 mg, 1.09 mmol) at 0° C. The reaction was stirred for 16 h at 50° C. The solvent was concentrated and residue was purified by prep-HPLC (Column: Boston Prime C18, 30×150 mm, 5 μm; Mobile Phase k 0.05% NH40H in water (v/v); Mobile phase B: 0.05% NH40H in MeCN (v/v); 16-46% B gradient, 8 min, hold at 100% B for 2 min; flow rate 30 mL/min, to provide the title compound (58.2 mg, 25.8%) as a white solid. LC/MS m/z (M+H)⁺=497.3; ¹H NMR (400 MHz, MeOD) δ 7.37 (d, 1H), 6.85 (s, 1H), 4.28 (q, 2H), 3.78 (s, 2H), 3.69 (s, 2H), 3.61 (s, 2H), 3.27 (s, 2H), 2.75 (s, 2H), 2.55 (s, 3H), 1.70 (s, 4H), 1.54 (t, 3H), 1.41 (s, 6H).

### Example 8: 1′-(7-ethoxy-3-methyl-1H-Indazole-5-carbonyl)-2-(1-hydroxy-2-methylpropan-2-yl)-5H-spiro[benzo[d]thiazole-6,4′-piperidin]-4(7H)-one M8 Metabolite

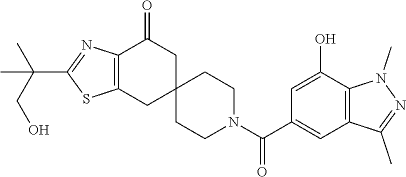 [SMILES: Cc1nn(C)c2c(O)cc(C(=O)N3CCC4(CC3)CC(=O)c3nc(C(C)(C)CO)sc3C4)cc12]

Prepared in the same manner as Example 5, using A12 (219 mg, 0.66 mmol) and ethyl 7-hydroxy-1,3-dimethyl-1H-indazole-5-carboxylate C6 (130 mg, 0.63 mmol) to give title compound (148.7 mg, 58.7%); LC/MS m/z (M+H)⁺=483.3; ¹H NMR (400 MHz, MeOD) δ 7.22 (d, 1H), 6.70 (d, 1H), 4.23 (s, 3H), 3.69 (s, 2H), 3.60 (s, 4H), 3.26 (s, 2H), 2.74 (s, 2H), 2.50 (s, 3H), 1.69 (s, 4H), 1.41 (s, 6H).

### Biological Evaluation

High content imaging assay to quantify lipid droplets in a human sebocyte cell line One week prior to cell dosing, SZ95 human sebocytes were thawed and grown in a T175 tissue culture flask containing 50 mL media. Media was prepared as follows: Sebomed basal medium with stable glutamine and without phenol red (Sigma; Catalog No. F8205), 10% heat inactivated fetal bovine serum (Invitrogen; Cat. No. 10082), 5 ng/mL recombinant human epidermal growth factor (Gibco; Catalog No. PHG0311), 1 mM calcium chloride (Fisher Scientific; Catalog No. BP9742), and 1X penicillin/streptomycin (Thermo Fisher; Catalog No. 15140-122). Cells were cultured at 37° C. and the media was replaced every 48-72 hours until start of assay. Compounds were delivered as a 75 nL spot by the Echo 550 (Labcyte) into 384-well assay plates (PerkinElmer; Catalog No. 6057308) with final compound concentrations of 10, 3.162, 1.000, 0.316, 0.100, 0.032, 0.0010, 0.003, 0.001, 0.0003, and 0.0001 μM. The final DMSO concentration was 0.1%. SZ-95 cells were washed with Dulbecco's phosphate buffered saline (DPBS, Lonza; Catalog No. 17-512Q) and then detached with 0.25% Trypsin-EDTA (Gibco; Catalog No. 25200056). Growth media (25 mL) was added to the flask and the cells were further diluted to 1.33×10∧5 cells/mL. SZ-95 cells were plated at a density of 10,000 cells/well in 75 μL and incubated for 48 hours at 37° C. Using the Biomek FX (Beckman), 25 μL of media was removed and the cells were fixed by adding 18.7 μL of 16% paraformaldehyde (Electron Microscopy Sciences; Cat. No. 50980488). After a 30 minute incubation at room temperature, the plates were washed twice with 75pi DPBS. Following the second wash, all remaining DPBS was removed. Staining solution was prepared with 2 μM Bodipy (Invitrogen; Cat. No D3922, diluted 1:1000) and Hoechst (Life Technologies; Cat. No. H3570, diluted 1:2000) in DPBS. Using the Biomek FX, 30 μL of the staining solution was added to each well. The cells were incubated for 20 minutes at room temperature, then washed once with 75 μL DPBS. Finally, 30 μL of DPBS was added to each well and the plates were sealed with light blocking film. The plates were read on an Opera Phenix (PerkinElmer) for high content imaging. Nuclei were detected by Hoechst staining and lipid droplets by Bodipy, which stains neutral lipids. Active compounds caused a reduction in the number and area of lipid droplets. The percent (%) effect at each concentration of compound is calculated by Genedata Screener analysis program using a four-parameter logistic dose response equation and is based on and relative to the amount of lipid droplets in the positive and negative control wells contained within each assay plate to determine the 50% inhibition concentration (IC50).

TABLE 2

| | | Human SZ95 Sebocyte |
|---|---|---|
| | | Lipid Imaging Assay / |
| | | Percent Inhibition of Lipid |
| Example | Structure | Droplets IC₅₀ (nM) |
| 1 | 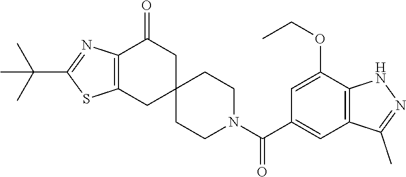 [SMILES: CCOc1cc(C(=O)N2CCC3(CC2)CC(=O)c2nc(C(C)(C)C)sc2C3)cc2c(C)n[nH]c12] | 10 |
| 2 | 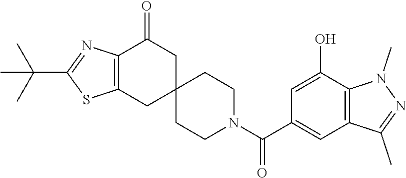 [SMILES: Cc1nn(C)c2c(O)cc(C(=O)N3CCC4(CC3)CC(=O)c3nc(C(C)(C)C)sc3C4)cc12] | 11 |
| 3 | 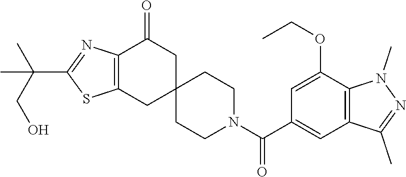 [SMILES: CCOc1cc(C(=O)N2CCC3(CC2)CC(=O)c2nc(C(C)(C)CO)sc2C3)cc2c(C)nn(C)c12] | 16 |
| 4 | 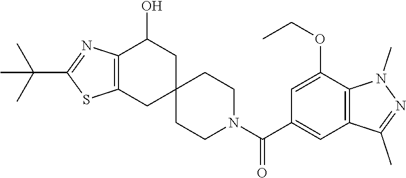 [SMILES: CCOc1cc(C(=O)N2CCC3(CC2)Cc2sc(C(C)(C)C)nc2C(O)C3)cc2c(C)nn(C)c12] | 20 |
| 4a | 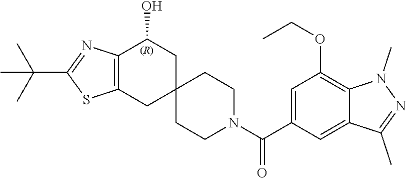 | 122 |
| 4b | 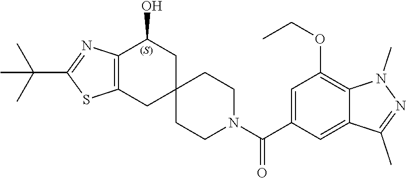 | 7 |
| 5 | 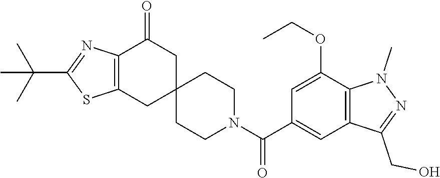 [SMILES: CCOc1cc(C(=O)N2CCC3(CC2)CC(=O)c2nc(C(C)(C)C)sc2C3)cc2c(CO)nn(C)c12] | ND |
| 6 | 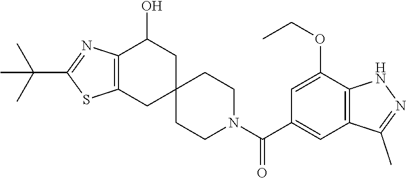 [SMILES: CCOc1cc(C(=O)N2CCC3(CC2)Cc2sc(C(C)(C)C)nc2C(O)C3)cc2c(C)n[nH]c12] | ND |
| 7 | 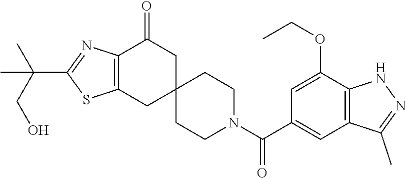 [SMILES: CCOc1cc(C(=O)N2CCC3(CC2)CC(=O)c2nc(C(C)(C)CO)sc2C3)cc2c(C)n[nH]c12] | ND |
| 8 | 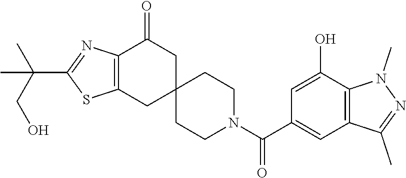 [SMILES: Cc1nn(C)c2c(O)cc(C(=O)N3CCC4(CC3)CC(=O)c3nc(C(C)(C)CO)sc3C4)cc12] | >0.145 |
| | 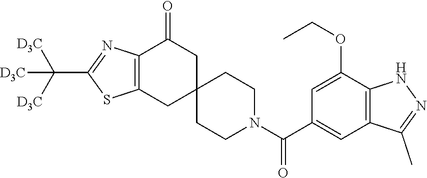 [SMILES: [2H]C([2H])([2H])C(c1nc2c(s1)CC1(CCN(C(=O)c3cc(OCC)c4[nH]nc(C)c4c3)CC1)CC2=O)(C([2H])([2H])[2H])C([2H])([2H])[2H]] | ND |
| | 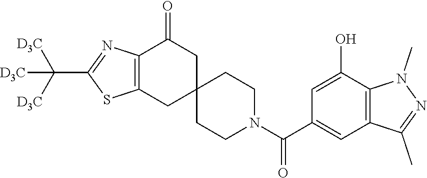 [SMILES: [2H]C([2H])([2H])C(c1nc2c(s1)CC1(CCN(C(=O)c3cc(O)c4c(c3)c(C)nn4C)CC1)CC2=O)(C([2H])([2H])[2H])C([2H])([2H])[2H]] | ND |
| | 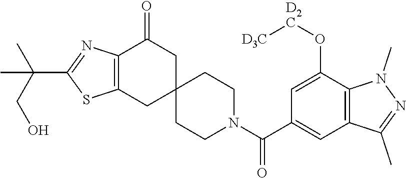 | ND |
| | 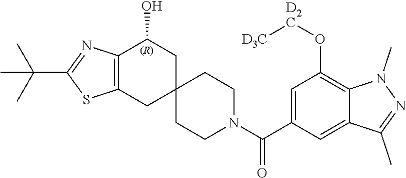 | ND |
| | 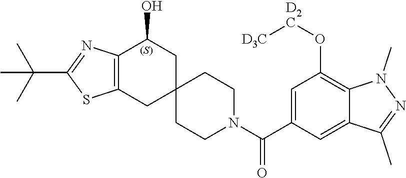 | ND |

1. A compound of formula I having the structure: 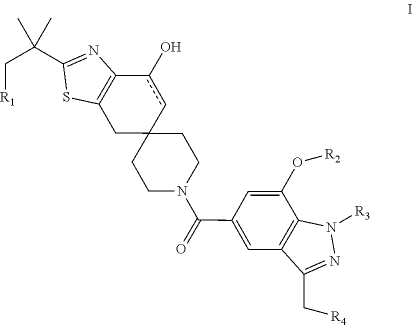
or a pharmaceutically acceptable salt, wherein R₁ is H or OH, R₂ is OH or ethyl, R₃ is H or methyl, and R₄ is H or OH; and, wherein the dashed line may be present or absent, and if present, indicates a double bond.

2. The compound of claim 1 or a pharmaceutically acceptable salt thereof, wherein R₁ is H, R₂ is ethyl; R₃ is H, and R₄ is H.

3. The compound of claim 1 or a pharmaceutically acceptable salt thereof, wherein R₁ is OH, R₂ is ethyl; R₃ is methyl, and R₄ is H.

4. A compound of formula I having the structure: 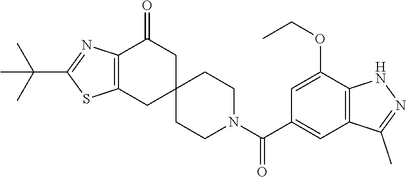 [SMILES: CCOc1cc(C(=O)N2CCC3(CC2)CC(=O)c2nc(C(C)(C)C)sc2C3)cc2c(C)n[nH]c12]
or a pharmaceutically acceptable salt thereof.

5. A compound of formula I having the structure: 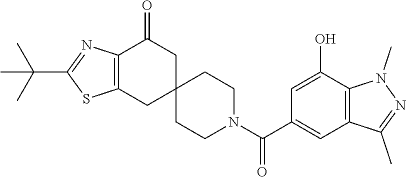 [SMILES: Cc1nn(C)c2c(O)cc(C(=O)N3CCC4(CC3)CC(=O)c3nc(C(C)(C)C)sc3C4)cc12]
or a pharmaceutically acceptable salt thereof.

6. A compound of formula I having the structure: 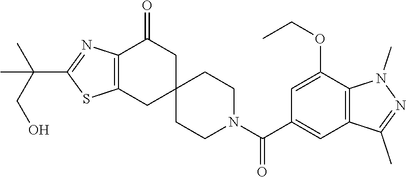 [SMILES: CCOc1cc(C(=O)N2CCC3(CC2)CC(=O)c2nc(C(C)(C)CO)sc2C3)cc2c(C)nn(C)c12]
or a pharmaceutically acceptable salt thereof.

7. A compound of formula I having the structure: 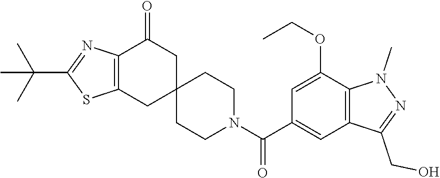 [SMILES: CCOc1cc(C(=O)N2CCC3(CC2)CC(=O)c2nc(C(C)(C)C)sc2C3)cc2c(CO)nn(C)c12]
or a pharmaceutically acceptable salt thereof.

8. The compound of claim 1 selected from the group consisting of:
2-(tert-butyl)-1′-(7-ethoxy-3-methyl-1H-indazole-5-carbonyl)-5H-spiro[benzo[d]thiazole-6,4′-piperidin]-4(7H)-one;
2-(tert-butyl)-1′-(7-hydroxy-1,3-dimethyl-1H-indazole-5-carbonyl)-5H-spiro[benzo[d]thiazole-6,4′-piperidin]-4(7H)-one;
1′-(7-ethoxy-1,3-dimethyl-1H-indazole-5-carbonyl)-2-(1-hydroxy-2-methylpropan-2-yl)-5H-spiro[benzo[d]thiazole-6,4′-piperidin]-4(7H)-one;
rac-(R)-(2-(tert-butyl)-4-hydroxy-4,7-dihydro-5H-spiro[benzo[d]thiazole-6,4′-piperidin]-1′-yl)(7-ethoxy-1,3-dimethyl-1H-indazol-5-yl)methanone;
2-(tert-butyl)-1′-(7-ethoxy-3-(hydroxymethyl)-1-methyl-1H-indazole-5-carbonyl)-5H-spiro[benzo[d]thiazole-6,4′-piperidin]-4(7H)-one;
rac-(2-(tert-butyl)-4-hydroxy-4,7-dihydro-5H-spiro[benzo[d]thiazole-6,4′-piperidin]-1′-yl)(7-ethoxy-3-methyl-1H-indazol-5-yl)methanone;
1′-(7-ethoxy-3-methyl-1H-indazole-5-carbonyl)-2-(1-hydroxy-2-methylpropan-2-yl)-5H-spiro[benzo[d]thiazole-6,4′-piperidin]-4(7H)-one;
1′-(7-hydroxy-3-methyl-1H-indazole-5-carbonyl)-2-(1-hydroxy-2-methylpropan-2-yl)-5H-spiro[benzo[d]thiazole-6,4′-piperidin]-4(7H)-one; or,
a pharmaceutically acceptable salt thereof.

9. The compound of claim 1 wherein the compound is 2-(tert-butyl)-1′-(7-ethoxy-3-methyl-1H-indazole-5-carbonyl)-5H-spiro[benzo[d]thiazole-6,4′-piperdin]-4(7H)-one; or, a pharmaceutically acceptable salt thereof.

10. The compound of claim 1 wherein the compound is 2-(tert-butyl)-1′-(7-hydroxy-1,3-dimethyl-1H-indazole-5-carbonyl)-5H-spiro[benzo[d]thiazole-6,4′-piperidin]-4(7H)-one; or, a pharmaceutically acceptable salt.

11. The compound of claim 1 wherein the compound is 1′-(7-ethoxy-1,3-dimethyl-1H-indazole-5-carbonyl)-2-(1-hydroxy-2-methylpropan-2-yl)-5H-spiro[benzo[d]thiazole-6,4′-piperidin]-4(7H)-one; or, a pharmaceutically acceptable salt.

12. The compound of claim 1 wherein the compound is rac-(R)-(2-(tert-butyl)-4-hydroxy-4,7-dihydro-5H-spiro[benzo[d]thiazole-6,4′-piperidin]-1′-yl)(7-ethoxy-1,3-dimethyl-1H-indazol-5-yl)methanone; or, a pharmaceutically acceptable salt.

13. The compound of claim 1 wherein the compound is 2-(tert-butyl)-1′-(7-ethoxy-3-(hydroxymethyl)-1-methyl-1H-indazole-5-carbonyl)-5H-spiro[benzo[d]thiazole-6,4′-piperidin]-4(7H)-one; or, a pharmaceutically acceptable salt.

14. The compound of claim 1 wherein the compound is rac-(2-(tert-butyl)-4-hydroxy-4,7-dihydro-5H-spiro[benzo[d]thiazole-6,4′-piperidin]-1′-yl)(7-ethoxy-3-methyl-1H-indazol-5-yl)methanone; or, a pharmaceutically acceptable salt.

15. The compound of claim 1 wherein the compound is 1′-(7-ethoxy-3-methyl-1H-indazole-5-carbonyl)-2-(1-hydroxy-2-methylpropan-2-yl)-5H-spiro[benzo[d]thiazole-6,4′-piperidin]-4(7H)-one; or, a pharmaceutically acceptable salt.

16. The compound of claim 1 wherein the compound is 1′-(7-hydrooxy-1,3-dimethyl-1H-indazole-5-carbonyl)-2-(1-hydroxy-2-methylpropan-2-yl)-5H-spiro[benzo[d]thiazole-6,4′-piperidin]-4(7H)-one; or, a pharmaceutically acceptable salt.

17. The compound or a pharmaceutically acceptable salt thereof according to any one of claims 1 to 16 in an isolated form.

18. The compound or a pharmaceutically acceptable salt thereof of claims 1 to 16, in crystalline form.

19. A pharmaceutical composition comprising a compound of claim 1, or a pharmaceutically acceptable salt thereof, and a pharmaceutically acceptable excipient.

20. A method of treating or preventing a disease or condition for which a ACC inhibitor is indicated, in a subject in need of such treatment, comprising administering to the subject a therapeutically effective amount of a compound of claim 1, or a pharmaceutically acceptable salt thereof.

21-30. (canceled)
